(19) United States
(12) Patent Application Publication
(10) Pub. No.: US 2025/0249065 A1
(43) Pub. Date: Aug. 7, 2025

(54) Application of Gastrodia elata Blume derived nano-extracellular vesicles in the preparation of drugs for the prevention and/or treatment of subarachnoid hemorrhage

(71) Applicant: Zhejiang University, Hangzhou (CN)

(72) Inventors: Anke ZHANG, Zhejiang (CN); Yan CHEN, Zhejiang (CN); Yibo LIU, Zhejiang (CN); Ling YUAN, Zhejiang (CN); Ting CHEN, Zhejiang (CN); Jianmin ZHANG, Zhejiang (CN)

(73) Assignee: Zhejiang University, Hangzhou (CN)

(21) Appl. No.: 18/822,591

(22) Filed: Sep. 3, 2024

(30) Foreign Application Priority Data
Feb. 1, 2024 (CN) 202410145026.9

(51) Int. Cl. A61K 36/8988; A61P 7/04

(52) U.S. Cl. CPC A61K 36/8988; A61P 7/04

(57) ABSTRACT

The invention belongs to the field of biomedicine technology and discloses the application of Gastrodia elata Blume derived nano-extracellular vesicles in the preparation of drugs for the prevention and/or treatment of subarachnoid hemorrhage (SAH). The Gastrodia elata Blume-derived nano-extracellular vesicles of this invention are obtained from separation of Gastrodia elata Blume by water extraction. The ingredients of this invention are natural, none of toxic side effects and with good biocompatibility and safety. Therefore, this invention has broad application prospects. The invention uses Gastrodia elata Blume-derived nano-extracellular vesicles to conduct in vitro and in vivo experiments and finds that it can inhibit the activation of microglia after subarachnoid hemorrhage by inhibition of the transformation of microglia into M1 phenotype and promoting the transformation of microglia into M2 phenotypic transformation which has significant effect in treating SAH, and can be used to prepare drugs for preventing and/or treating subarachnoid hemorrhage.

### CROSS-REFERENCE TO RELATED APPLICATIONS

This is a U.S. patent application which claims the priority and benefit of Chinese Patent Application Number 2024101450269, filed on Feb. 1 2024, the disclosure of which is incorporated herein by reference in its entirety.

### TECHNICAL FIELD

The invention belongs to the field of biomedicine technology and discloses the application of Gastrodia elata Blume derived nano-extracellular vesicles in the preparation of drugs for the prevention and/or treatment of subarachnoid hemorrhage (SAH).

### BACKGROUND

Due to the complexity of the brain, the treatment of neurodegenerative diseases has always been a clinical problem. The main reason lies in the difficulty of tissue regeneration and persistent neuroinflammation. Subarachnoid hemorrhage (SAH) is a devastating cerebrovascular disease, resulting in high mortality and morbidity. In recent studies, it has been found that brain damage caused by acute inflammation accompanying early subarachnoid hemorrhage may be the main reason for the poor prognosis of SAH patients. During the process of SAH, thrombosis caused by subarachnoid hemorrhage and the release of blood contents, resulting in ischemic and inflammatory reactions, are the main causes of acute inflammation. However, currently clinical treatment strategies for acute inflammation and early brain injury are still lacking in modern medicine. Although for many years the central nervous system was considered an immune-privileged organ, it is now generally accepted that there are interconnections between the immune and nervous systems. Furthermore, accumulating data indicate that in the brain, inflammatory cells are involved in tissue remodeling after injury. Microglia are the resident macrophages of the brain and play an important role as resident immune and phagocytes in the central nervous system. Ekdahl and colleagues reported an increase in the number of activated microglia 16 weeks after MCAO in rats. Microglia can transform into phagocytes upon activation by ischemia and release a variety of substances, many of which have cytotoxic and/or cytoprotective effects. Microglia may produce neurotrophic molecules.

Such as brain-derived neurotrophic factor (BDNF), insulin-like growth factor I (IGF-I) and several other growth factors to exert neuroprotective effects. A large amount of evidence suggests that activated microglia may release some pro-inflammatory cytokines, such as TNF-α, IL-1β, and IL-6, as well as other potential cytotoxic molecules, including NO, ROS, and prostaglandins during SAH. Excitotoxicity and oxidative stress caused by SAH activate microglia and astrocytes, and these cells react by secreting cytokines, chemokines, and matrix metalloproteases (MMPs). These inflammatory mediators lead to the upregulation of endothelial cell adhesion molecules, allowing blood-borne inflammatory cells (mainly neutrophils) to infiltrate ischemic brain areas. Neutrophils also secrete cytokines, leading to further activation of glial cells. These processes can lead to neuronal cell death and enhance damage to the ischemic brain. Under pathological conditions, microglia are rapidly activated and accompanied by transcriptional adaptive functional changes. Activated microglia have two phenotypes, M1 and M2. When exposed to endotoxins (such as LPS), 1-methyl-4-phenyl-1,2,3,6-tetrahydropyridine (MPTP), 6-hydroxydopamine (6-OHDA), alpha-synuclein, brain injury and as well as other stimuli, the M1 phenotype of microglia is activated and secretes a large amount of pro-inflammatory cytokines such as IL-1β, IL-6 and TNF-α to mediate damage to neuronal cells; while the neuronal damage or death will in turn further activate M1 microglia, forming a vicious cycle and exacerbating the pathological process. At the same time, when microglia are stimulated, intracellular enzymes such as oxidase, cyclooxygenase-2 (COX-2), inducible nitric oxide synthase (iNOS), etc. are activated to varying degrees and release a large amount of Inflammatory mediators such as ROS, NO, etc. can cause neuronal damage or even death. Therefore, M1 microglia are considered to be the main initiators and participants of inflammatory responses in the brain. M2-type microglia are divided into three subtypes: M2a, M2b and M2c. It is generally believed that M2-type microglia play a protective role in neuronal cells and secrete anti-inflammatory factors (IL-10) and transforming growth factors-β (TGF-β) and neurotrophic factors (BDNF, GDNF), etc., It plays the role of inhibiting immune inflammatory response and promoting tissue repair. Although M1 type and M2 type microglia have different biological functions, after brain injury, they respectively express unique cell markers and jointly exert effects on the brain. Among them, selective activation of M2 phenotype microglia in tissues can effectively participate in the repair of acute brain injury. In addition, a large amount of evidence illustrates its important role in the early inflammatory response after SAH. It has also been reported that M2 microglia can promote hematoma clearance, neuronal survival and alleviate brain damage. Therefore, targeted drugs that activate M2 microglia have become a new therapeutic strategy to prevent early brain damage in SAH.

Exosomes are small vesicles with a diameter of 30-150 nm secreted by cells. They have a typical lipid bilayer structure. They are present in biological fluids such as cell culture supernatant, serum, and plasma, and contain nucleic acids and proteins. lipids and other inclusions, participating in various biological functions such as molecular transmission between cells, immune response, cell migration, differentiation and so on. In recent years, studies have shown that plants can also produce exosomes and have various biological functions such as anti-inflammatory, regulating intestinal flora, and participating in lipid metabolism.

Gastrodia elata Blume has the effects of sedation, anti-convulsant, blood pressure lowering, anti-myocardial ischemia, anti-arrhythmia, anti-inflammation, anti-aging, antioxidant, anti-radiation, blood vessel dilation, anti-coagulation, and anti-thrombosis. The function of neuron damage is widely used in traditional Chinese medicine. However, due to the existence of the blood-brain barrier, the current drug action mechanism for Gastrodia elata Blume after SAH and related nerve damage currently cannot meet the relevant mechanism description.

Therefore, in-depth research on the mechanism of action of Gastrodia elata Blume after SAH, and the development of new Gastrodia elata Blume active ingredient preparations will provide new theoretical foundations and research ideas for clinical treatment and drug application of SAH.

### SUMMARY

In order to overcome the shortcomings and deficiencies of the above-mentioned prior art, the primary purpose of the present invention is the application of Gastrodia elata Blume derived nano-extracellular vesicles in the preparation of drugs for the prevention and/or treatment of subarachnoid hemorrhage.

The Gastrodia elata Blume-derived nano-extracellular vesicles of the present invention have the functions of inhibiting microglial activation induced by subarachnoid hemorrhage, inhibiting the transformation of microglia to M1 phenotype, and promoting the transformation of microglia to M2 phenotype. It has significant effect in treating SAH, and can be used to prepare drugs for preventing and/or treating subarachnoid hemorrhage:

The object of the invention is achieved by the following scheme:

Application of Gastrodia elata Blume Extracellular Vesicles (RGEV) in the preparation of drugs for the prevention and/or treatment of subarachnoid hemorrhage.

The application of Gastrodia elata Blume derived nano-extracellular vesicles in the preparation of drugs for preventing subarachnoid hemorrhage.

The application of Gastrodia elata Blume derived nano-extracellular vesicles in the preparation of drugs for the treatment of subarachnoid hemorrhage.

In the present invention, the diameter of the Gastrodia elata Blume derived nano-extracellular vesicles is 50-250 nm.

The same or different medicaments respectively include a therapeutically effective amount of Gastrodia elata Blume-derived nano-extracellular vesicles.

The same or different described medicines can be made into various pharmaceutical dosage forms using conventional methods, and these dosage forms include: tablets, sugar-coated tablets, thin-film coated tablets, enteric coated tablets, capsules, hard capsules, soft capsules, oral liquid, oral agent, granules, granules, granules, pills, Dan, suspension, wine, tincture, drops and as well as dosage form and injection for oral administration or dosage forms other than oral, such as injections.

The same or different described drugs may also contain one or more pharmaceutically acceptable carriers or excipients.

The carrier or auxiliary material may include diluents, adhesives, surfactants, humectants, adsorption carriers, lubricants, fillers, disintegrants, preservatives, etc.

The present invention also provides the application of the above-mentioned Gastrodia elata Blume derived nano-extracellular vesicles in the preparation of products that promote the transformation of microglia to M2 phenotype.

The present invention also provides the application of the above-mentioned Gastrodia elata Blume derived nano-extracellular vesicles in the preparation of products that inhibit the transformation of microglia to M1 phenotype.

The present invention also provides the application of the above-mentioned Gastrodia elata Blume derived nano-extracellular vesicles in the preparation of products that inhibit microglial activation induced by subarachnoid hemorrhage.

Further, the product can be a drug.

In the present invention, the Gastrodia elata Blume derived nano-extracellular vesicles are isolated from Gastrodia elata Blume.

Gastrodia elata Blume-derived nano-extracellular vesicles of the present invention are obtained by water extraction and separation of Gastrodia elata Blume. They have natural ingredients, no toxic side effects, good biocompatibility and safety, and therefore have broad application prospects.

The present invention utilizes nanometer extracellular vesicles derived from Gastrodia elata Blume to conduct in vitro and in vivo experiments, and finds that it has the ability to inhibit microglial activation induced by subarachnoid hemorrhage, inhibit the transformation of microglia to M1 phenotype, and promote microglia. The activity of cells transforming into M2 phenotype has a significant effect in treating SAH and can be used to prepare drugs for preventing and/or treating subarachnoid hemorrhage.

### BRIEF DESCRIPTION OF DRAWINGS

In order to more clearly illustrate the technical solutions of the embodiments of the present invention, the accompanying drawings required to be used in the embodiments will be briefly introduced below. It should be understood that the following accompanying drawings only illustrate some embodiments of the present invention. Therefore, it should not be regarded as limiting the scope. For those of ordinary skill in the art, other relevant drawings can also be obtained based on these drawings without exerting creative efforts.

FIG. 1 is a schematic diagram of the extraction and separation of RGEV.

FIG. 2 is a transmission electron microscope image of RGEV.

FIG. 3 is a particle size diagram of RGEV.

FIG. 4 is a protein quantitative curve diagram of RGEV.

FIG. 5 is a gel image of RGEV.

FIG. 6 is a diagram of the uptake of RGEV by microglial BV2.

FIG. 7-FIG. 8 is a diagram showing the influence of REGV on BV2 cell inflammatory factor secretion and M1, M2 phenotype under the action of LPS. Wherein, the data in the figure *P<0.05; **P<0.01; ***P<0.001.

FIG. 9 is the in vivo distribution fluorescence diagram of RGEV.

FIG. 10 is a diagram of the brain damage repair of SAH model rats by RGEV.

FIG. 11 is the impact of RGEV on IL-1beta expression in brain tissue of SAH model rats and the changes in macrophages.

### DETAILED DESCRIPTION

The present invention will be described in further detail below in conjunction with the examples, but the embodiments of the present invention are not limited thereto. The materials involved in the following examples can be obtained from commercial sources unless otherwise specified. The methods described are conventional methods unless otherwise specified.

In one embodiment, the use of Gastrodia elata Blume Extracellular Vesicles (RGEV) in the preparation of drugs for the prevention and/or treatment of subarachnoid hemorrhage.

In one embodiment, the application of Gastrodia elata Blume derived nano-extracellular vesicles in the preparation of drugs for preventing subarachnoid hemorrhage.

One embodiment is the application of Gastrodia elata Blume derived nano-extracellular vesicles in the preparation of drugs for the treatment of subarachnoid hemorrhage.

In one embodiment, the diameter of the Gastrodia elata Blume-derived nano-extracellular vesicles is 50-250 nm.

In one embodiment, the above-mentioned Gastrodia elata Blume derived nano-extracellular vesicles are used in the preparation of products that promote the transformation of microglia to M2 phenotype.

In one embodiment, the above-mentioned Gastrodia elata Blume derived nano-extracellular vesicles are used in the preparation of products that inhibit the transformation of microglia to M1 phenotype.

In one embodiment, the above-mentioned Gastrodia elata Blume derived nano-extracellular vesicles are used in the preparation of products that inhibit microglial activation induced by subarachnoid hemorrhage.

In one embodiment, the product may be a drug.

In one embodiment, the same or different medicines respectively comprise a therapeutically effective amount of Gastrodia elata Blume-derived nano-extracellular vesicles.

The dosage of the medicine or pharmaceutical composition of the present invention is determined according to the formulation method, administration method, patient's age, weight, gender, disease condition, diet, administration time, administration route, excretion speed and reaction sensitivity. A variety of prescriptions may be made based on such factors. A skilled physician can usually readily determine the prescription and dosage effective for the desired treatment or prophylaxis.

Described therapeutically effective dose refers to the amount that can produce therapeutic effect to humans and/or animals and can be accepted by humans and/or animals. For example, a therapeutically or pharmaceutically effective amount of a drug is the amount of drug required to produce the desired therapeutic effect, which may be reflected by the results of clinical trials, model animal studies, and/or in vitro studies. The pharmaceutically effective dose depends on several factors, including but not limited to characteristics of the treatment subject (such as the height, weight, gender, age and medication history of the treatment subject), and the severity of the disease.

In specific embodiments of the present invention, the therapeutically effective amount refers to an amount that can produce preventive and/or therapeutic effects on SAH patients and can be accepted by the patient.

In specific embodiments of the invention, the modes of administration of the drug or drug composition include but are not limited to oral administration, non-gastrointestinal administration, inhalation spray administration, local administration, rectal administration, nasal administration, buccal administration or through an implanted drug storage device. Oral administration or injection is preferred.

In specific embodiments of the present invention, any orally acceptable dosage form may be used, including but not limited to capsules, tablets, aqueous suspensions or solutions.

In specific embodiments of the invention, liquid dosage forms for oral administration include, but are not limited to, pharmaceutically acceptable emulsions, microemulsions, solutions, suspensions, syrups and elixirs.

In specific embodiments of the invention, solid dosage forms for oral administration include capsules, tablets, pills, powders and granules.

In specific embodiments of the present invention, the medicine or pharmaceutical composition also includes a pharmaceutically acceptable carrier or auxiliary material. The pharmaceutically acceptable carrier or excipient may contain inert ingredients that do not unduly inhibit the biological activity of the compound. A pharmaceutically acceptable carrier or excipient should be biocompatible, eg, non-toxic, non-inflammatory, non-immunogenic or have no other undesirable reactions or side effects when administered to a subject. Standard drug formulation techniques can be used.

Pharmaceutically acceptable carriers or auxiliary materials include but are not limited to diluents, adhesives, surfactants, humectants, adsorption carriers, lubricants, fillers, disintegrants, preservatives, etc. These substances are used as necessary to aid the stability of the formulation or to help enhance the activity or its biological effectiveness or to produce an acceptable taste or odor in the case of oral administration. The preparation that may be used in such pharmaceutical composition may be in the form of its original compound itself or optionally in the form of its pharmacologically acceptable salt. Drug compositions so formulated may be administered in any appropriate manner known to a person skilled in the art, if necessary.

Wherein, diluent includes but is not limited to lactose, sodium chloride, glucose, urea, starch, water.

Binders include but are not limited to starch, pregelatinized starch, dextrin, maltodextrin, sucrose, gum arabic, gelatin, methyl cellulose, carboxymethyl cellulose, ethyl cellulose, polyvinyl alcohol, polyethylene glycol, polyvinylpyrrolidone, alginic acid and alginate, xanthan gum, hydroxypropyl cellulose and hydroxypropyl methylcellulose.

Surfactant includes but is not limited to polyoxyethylene sorbitan fatty acid ester, sodium lauryl sulfate, stearic acid monoglyceride, cetyl alcohol.

Humectants include but are not limited to glycerin and starch.

Adsorption carriers include, but are not limited to, starch, lactose, bentonite, silica gel, kaolin, and bentonite.

Lubricant includes but is not limited to zinc stearate, glyceryl monostearate, polyethylene glycol, talc, calcium and magnesium stearate, polyethylene glycol, boric acid powder, hydrogenated vegetable oil, stearyl rich Sodium malate, polyoxyethylene monostearate, monolauryl sucrose, sodium lauryl sulfate, magnesium lauryl sulfate, magnesium lauryl sulfate.

Filling agents include but are not limited to mannitol (granular or powdered), xylitol, sorbitol, maltose, erythrose, microcrystalline cellulose, polymeric sugar, coupled sugar, glucose, lactose, sucrose, dextrin, starch, sodium alginate, laminarin powder, agar powder, calcium carbonate and sodium bicarbonate.

Disintegrants include but are not limited to cross-linked vinylpyrrolidone, sodium carboxymethyl starch, low-substituted hydroxypropylmethyl, cross-linked sodium carboxymethyl cellulose, and soybean polysaccharide.

In specific embodiments of the present invention, the medicine or pharmaceutical composition also includes other medicines for preventing and/treating SAH, that is, the Gastrodia elata Blume derived nano-extracellular vesicles can be combined with other compounds that can be used for preventing and/treating SAH.

In one embodiment, the Gastrodia elata Blume-derived nano-extracellular vesicles are separated from Gastrodia elata Blume through conventional separation methods.

In one embodiment, the nano-extracellular vesicles derived from Gastrodia elata Blume are extracted and separated by crushing or breaking the walls of Gastrodia elata Blume and adding an extractant.

In one embodiment, the separated precipitate is Gastrodia elata Blume-derived nano-extracellular vesicles.

In one embodiment, the extraction agent is conventionally used water or an aqueous solution adding a buffer such as phosphate.

In one embodiment, the separation method is high-speed centrifugal separation.

In one embodiment, the speed of the high-speed centrifugation is 100000 g or above.

In one embodiment, the time of high-speed separation is 10-100 min.

In one embodiment, the time of described high-speed centrifugation is 60-100 min.

In one embodiment, the precipitate obtained by high-speed centrifugation can be resuspended in a buffer such as PBS to obtain a suspension of nanometer extracellular vesicles derived from Gastrodia elata Blume. One or more high-speed centrifugation can be performed to obtain further purified Gastrodia elata Blume-derived nano-extracellular vesicles.

In one embodiment, pre-centrifugation is performed to remove impurity precipitates before high-speed centrifugation.

In one embodiment, the speed of the pre-centrifugation is 10000 g or less. Pre-centrifugation can separate and remove non-target substances, reducing the impact of impurities on subsequent centrifugation.

In one embodiment, the time of described pre-centrifugation is 10-60 min. One or more pre-centrifugations can be performed.

In one embodiment, at least one centrifugation speed is performed at 8000-10000 g.

In one embodiment, filtering is performed before the pre-centrifugation, such as using gauze to filter the insoluble sediment.

In one embodiment, the Gastrodia elata Blume-derived nano-extracellular vesicle suspension is filtered with a filter before use to obtain a sterile Gastrodia elata Blume-derived nano-extracellular vesicle suspension.

In one embodiment, the filter is a 0.22 μm filter.

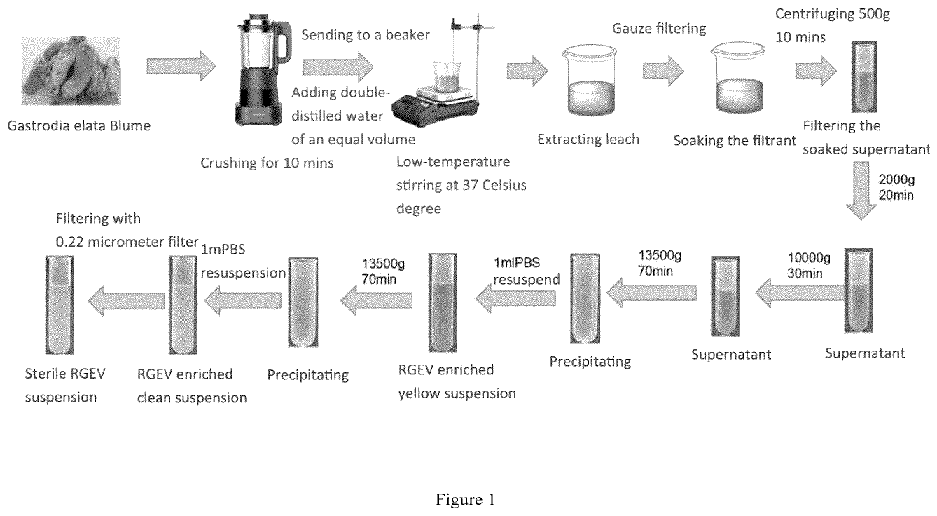

In one embodiment, the nanometer extracellular vesicles derived from Gastrodia elata Blume are obtained by extracting and separating Gastrodia elata Blume. The schematic diagram of the extraction process is shown in FIG. 1. Specifically, it is prepared by the following steps: after crushing or pulverizing Gastrodia elata Blume, add water and soak, separate the supernatant, pre-centrifuge the supernatant at a centrifugal force of 10000 g or below, take the supernatant, and discard the precipitate; the pre-centrifuged supernatant is then centrifuged at a high speed at a centrifugal force of 100000 g or above, and the centrifugation time can be 10-100 min, the resulting precipitate is Gastrodia elata Blume-derived nano-extracellular vesicles.

In one embodiment, the Gastrodia elata Blume is cleaned before use.

In one embodiment, the soaking time can be 6-24 h.

In one embodiment, stirring can be carried out during the soaking process.

In one embodiment, the supernatant liquid obtained by the separation can be obtained by filtration separation, such as using gauze to filter the insoluble sediment.

In one embodiment, one or more pre-centrifugations can be performed; such as centrifuging for 10-60 min respectively at 500-1000 g, 1500-2500 g, 8000-10000 g, taking the supernatant respectively, and discarding the precipitate.

In one embodiment, at least one centrifugation speed is performed at 8000-10000 g.

In one embodiment, the precipitate obtained by high-speed centrifugation can be resuspended in a buffer such as PBS to obtain a suspension of nanometer extracellular vesicles derived from Gastrodia elata Blume.

In one embodiment, multiple high-speed centrifugations can be performed. For example, the precipitate obtained by high-speed centrifugation is resuspended in sterile PBS and then centrifuged again at high speed.

In one embodiment, the Gastrodia elata Blume-derived nano-extracellular vesicle suspension is filtered with a filter before use to obtain a sterile Gastrodia elata Blume-derived nano-extracellular vesicle suspension.

In one embodiment, the filter is a 0.22 μm filter.

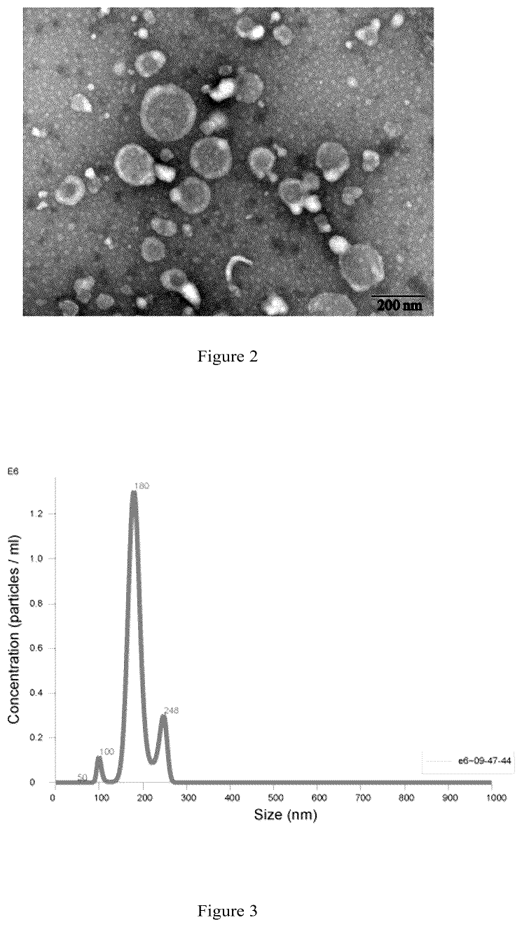

In one embodiment, transmission electron microscopy is used to conduct morphological identification of RGEV, and a nanoparticle tracking analyzer is used to measure the particle size of RGEV in the suspension. The results are shown in FIGS. 2 and 3. It can be seen from the figures that RGEV appears as round or oval vesicles, like a tray, with diameters ranging from 50 to 250 nm.

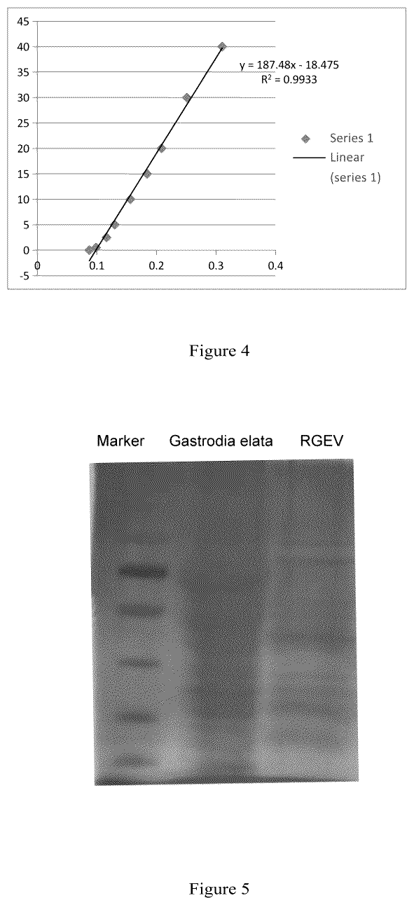

In one embodiment, the protein concentration of RGEV was quantitatively measured using a BCA detection kit (Thermo Scientific Company), the absorbance at a wavelength of 562 nm was measured, and the protein concentration in the suspension was measured to be 7.89 μg/μL. The results are shown in FIG. 4.

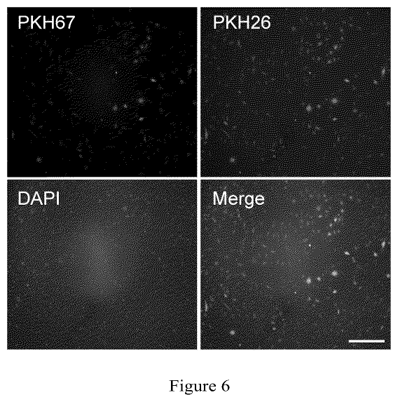

In one embodiment, the protein of RGEV suspension was identified by Coomassie blue staining. The results are shown in FIG. 5.

Embodiment 1: Identification of the uptake of RGEV by microglial BV2. (1) Incubation of PKH26/PKH27-RGEV: 10 mg PKH26/PKH27 and 10 particle number RGEV are incubated, protected from light at 37° C. Incubate for 30 minutes to fully combine RGEV with PKH26/PKH27; (2) Remove free PKH26/PKH27: Centrifuge the above mixture at high speed again with a centrifugal force of 130000 g for 70 minutes. Discard the supernatant, take the precipitate, and resuspend it in 1 mL PBS to obtain PKH26/PKH27-RGEV; (3) Cell incubation: Inoculate vigorously growing BV2 in a 6-well plate. When the cell confluence reaches 50-60%, add 10 uLPKH26/PKH27-RGEV and incubate at 37° C. in the dark for 6 hours; (4) DAPI nuclear staining: wash 2-3 times with PBS, add 2 mL of basic 1640 culture medium to each well, and add 10 μL of 1 mg/mL DAPI, put it into an incubator and incubate for 30-40 min; (5) On-machine detection: Fluorescence microscope (Sunny), take pictures at different magnifications, the results are shown in FIG. 6. As can be seen from the figure, PKH26/PKH27 RGEV can be taken up by BV2 cells and emit red/green fluorescence intracellularly, while no fluorescence was observed in the PBS control group. This result confirms that PKH26/PKH27 RGEV can be taken up into the cells by BV2 cells.

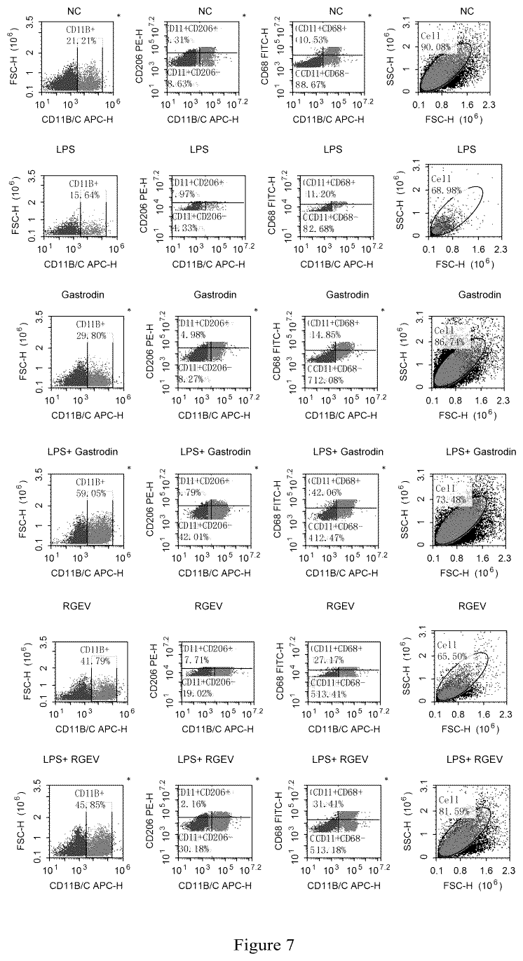

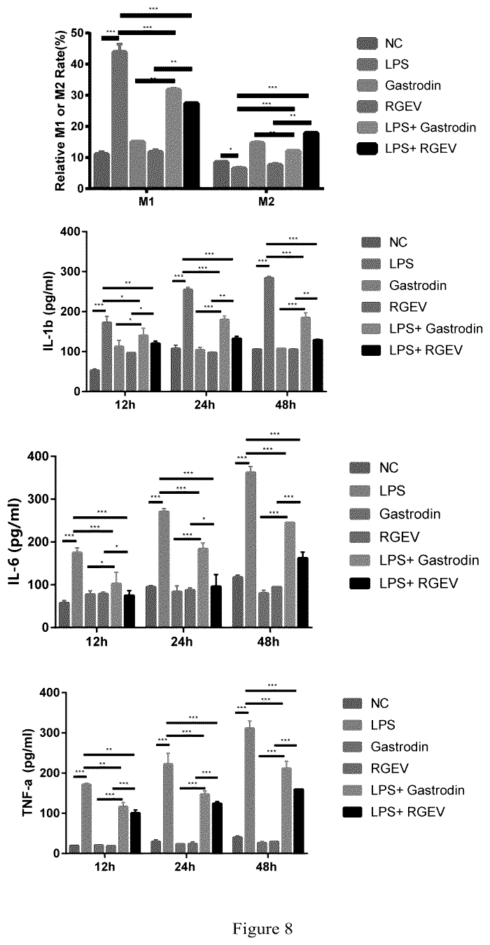

Embodiment 2: RGEV influences BV2 cell inflammatory factor secretion and M1, M2 phenotype under the action of LPS. (1) Experimental grouping: NC group, LPS group, Gastrodin group, LPS+Gastrodin group, RGEV group, LPS+RGEV group. BV2 cells were purchased from the Shanghai Cell Bank of the Chinese Academy of Sciences. (2) Model establishment: BV2 cells in good growth status were digested with trypsin, and digestion was terminated with complete culture medium. Centrifuge at 1200 rpm for 5 min to remove the supernatant. Add an appropriate amount of culture medium, resuspend the cell pellet, take an appropriate amount of cell suspension and count it with a cell counter to prepare a cell suspension of 2×10 cells/mL; spread the cell suspension into the well plate at a rate of 2 mL/well, place the paved 6-well plate in an incubator for normal culture; add endotoxin (LPS) at a final concentration of 100 μg/mL to stimulate BV2 cells to induce BV2 phenotype transformation to form a BV2 inflammatory injury model. (3) Experimental steps: After LPS induces BV2 cells for 12 h, each group adds the corresponding reagent, the LPS+gastrodin group, adds the gastrodin with a final concentration of 30 μmol/L, and continues to culture for 24 h; the LPS+RGEV group, Add RGEV, quantitate based on the protein concentration of RGEV, with the final concentration being 100 μg/mL, and continue culturing for 24 hours; after the culture is completed, digest and harvest the cells; (4) Result detection: 1×10 cells were taken for flow cytometry staining to detect M1 and M2 macrophages. Use FcRblock to incubate for 5-10 minutes at 4° C., add M1 macrophage flow cytometry antibody CD86 surface staining, and incubate at 4° C. for 30 minutes. Wash the cells 2-3 times with PBS, add cell fixation buffer (Fixation Buffer, 500 μL, 420801, BioLegend) and fix at room temperature for 30 minutes in the dark. Centrifuge at 150 g for 5 min, discard the fixative, add 2 mL of 10× Intracellular Staining Permeabilization Wash Buffer (421002, BioLegend) diluted 10 times with ddHO to resuspend the cells, centrifuge at 150 g for 5 min, discard the supernatant, and repeat the steps 2-3 times. Resuspend the cells in 100 μL 1×Intracellular Staining Permeabilization Wash Buffer, add M2 macrophage flow cytometry antibody CD206, and incubate at room temperature in the dark for 30 minutes. After incubation, wash the cells 2-3 times with 2 mL 1×Intracellular Staining Permeabilization Wash Buffer, add 500 μL cell staining buffer to resuspend the cells, and put the cells on the machine. FlowJo software analyzes the final flow cytometry results. The results are shown in FIGS. 7 and 8. (5)

As can be seen from the figure, flow cytometry technology detects changes in the M1 and M2 phenotypes of BV2 cells. It can be concluded that LPS can activate the M1 phenotype transformation of BV2, and the proportion of M1 cells increases significantly relative to the NC group. There is a difference between the two. Significant statistical difference; simple gastrodin and RGEV stimulation can reduce the differentiation of M1, and there is a statistical difference between the two groups compared with the NC group; simple gastrodin and RGEV stimulation can activate the differentiation of M2, and there is a statistical difference between the two groups compared with the NC group. LPS-induced differentiation of BV2 to M1 phenotype can be inhibited by gastrodin and RGEV, and increases the differentiation of M2 phenotype. At the same time, RGEV is more effective than gastrodin, and there is a significant statistical difference between the two groups; at the level of cellular inflammation LPS can induce the secretion of inflammatory factors IL-1b, IL-6, and TNF-α that activate BV2 cells, with statistically significant differences; gastrodin and RGEV have no significant regulatory effect on inflammatory factors, and there is no significant statistical difference compared with the NC group. Difference; gastrodin and RGEV can inhibit the increased secretion of inflammatory factors induced by LPS, and RGEV has a better effect than gastrodin, with a statistically significant difference.

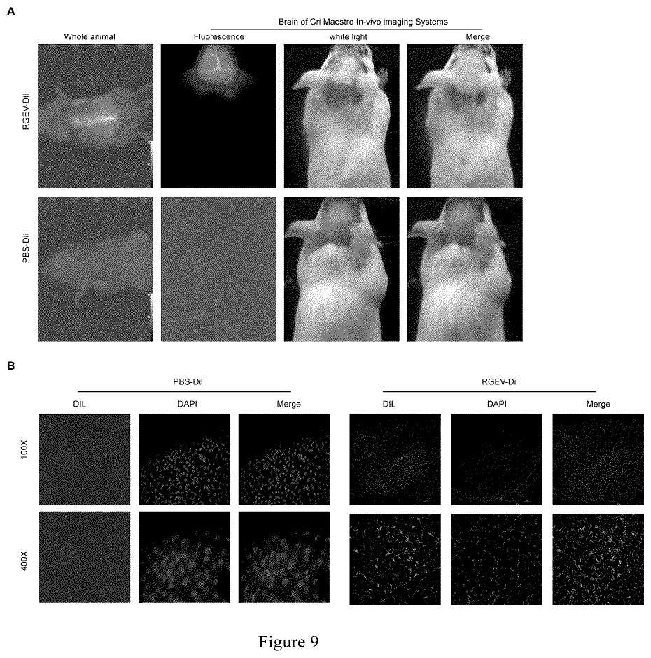

Embodiment 3: In vivo distribution of Gastrodia elata Blume derived nano-extracellular vesicles (RGEV). (1) Experimental subject: 8-week-old male SD rats; (2) (2) Experimental grouping: (1) PBS intragastric administration; 2 DIL-RGEV intraperitoneal injection (dose: 100 μg/rat), 3 rats in each group; (3) Incubation of DIL fluorescent probe: 10 mg DIL/PKH26/PKH27 and RGEV with 10 particles were incubated together, and incubated at 37° C. in the dark for 30 minutes to fully combine RGEV with DIL/PKH26/PKH27; use PBS Incubate with DIL as a negative control. (4) Remove free DIL/PKH26/PKH27: The above mixture is centrifuged again at high speed, centrifugal force 130000 g, centrifuge for 70 min, discard the supernatant, take the precipitate, and resuspend with 1 mL PBS. Obtain DIL/PKH26/PKH27-RGEV; (5) Experimental steps: Mice were fasted and water-deprived for 8 hours, grouped according to the experiment, and the mice were given intraperitoneal injection configuration 6 hours in advance. (6) According to the DIL-marked RGEV of the experimental group, after 6 hours, the brains were dissected and taken out, observed and photographed on an in vivo imager, and then the brains were cut into paraffin sections and observed and photographed under a fluorescence microscope. The results are shown in FIG. 9. (7) Result analysis: It can be seen from the figure that after intraperitoneal injection of DIL-RGEV, it can pass through the blood-brain barrier and be enriched in the brain. In the in vivo imaging of the rat brain, fluorescence can be seen It is enriched in the brain of rats, as shown in FIG. 9A; further dissection of the rat brain tissue and in vitro secondary imaging showed that there are fluorescence divisions in the rat brain, indicating that DIL-RGEV is in the brain It is enriched and taken up by the brain tissue into the cells, see FIG. 9B.

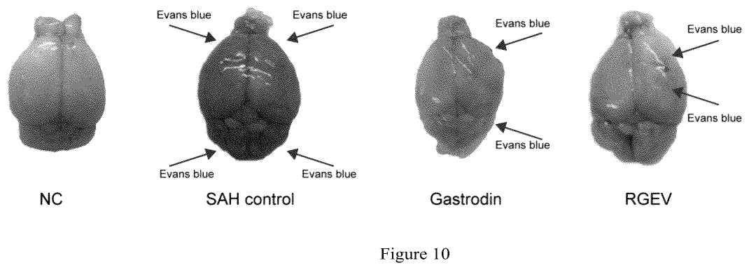

Embodiment 4: The situation of brain damage repair of SAH model rats by RGEV. (1) Experimental subjects: 8-week-old male SD rats; (2) Experimental grouping: (1) NC group; (2) SAH surgical model group; 33) Gastrodin+SAH surgical model group; ARGEV+SAH surgery model group, 3 rats in each group; (3) Experimental steps: After weighing, the rats were anesthetized intraperitoneally with 10% chloral hydrate at 0.4 g/kg. After the anesthesia was successful, the hair on the top of the skull was shaved and placed prone. position, fix the animal's head on the brain stereotaxic instrument, keeping the top of the skull in a horizontal position. The surgical area is routinely disinfected. A 2 cm-long skin incision is made along the mid-sagittal line of the cranium. Blunt separation of the muscles and periosteum is performed. Hydrogen peroxide is used to disinfect and stop bleeding. Apply 5 mL of water 6 mm in front of the midline of the bregma and 2-3 cm lateral to the midline. Drill the hole with the syringe needle, and use a No. 4 needle to carefully rupture the meninges under the operating microscope. When clear cerebrospinal fluid flows out, tilt the catheter forward 30° in the sagittal plane and insert it until the tip reaches the bottom of the anterior cranial fossa, and the depth is about 30 degrees from the surface of the brain. 1.0 cm. The bone hole is sealed with bone wax. Connect the syringe and aspirate gently. After seeing clear cerebrospinal fluid flowing out, it is confirmed that it has entered the subarachnoid space. After local disinfection, cut off the rat tail about 2 cm long, quickly collect 300 μL of rat tail artery blood, and inject it into the subarachnoid space with a microinjection for 20 seconds. Pull out the catheter, seal the bone hole with medical biological glue, suture the skin layer by layer, and place it in an incubator. After the rats recovered from anesthesia, they were placed back in the breeding box and observed. The animals in the administration group were administered 24 hours in advance. The dosage for the rats in the gastrodin group was calculated based on 6 times the human dosage, which is about 30 mg/kg; the dosage for the rats in the RGEV group was based on the exosome BCA quantification results and the protein concentration was 30 mg/kg. Drug; intraperitoneally administered once every 8 hours; subarachnoid hemorrhage. Rats were sacrificed 48 hours later, samples were taken, and Evan's blue staining was performed to compare the bleeding in the brains of each group; the results are shown in FIG. 10; (4) Experimental results: As can be seen from the figure, in the SAH surgical model group, it can be seen that the entire rat brain has been colored blue, and it can be concluded that the SAH model was successfully modeled, and the bleeding of the model group was more serious at the same time; the gastrodin group It can be seen that the bleeding is controlled around the modeling hole, and at the same time, the bleeding appears deep staining around the modeling hole, and nearly ⅔ of the bleeding staining occurs on the side of the modeling hole; RGEV has the best bleeding control effect, and it only occurs around the modeling hole. The results showed that RGEV administration in advance can effectively control bleeding damage after modeling and accelerate the absorption of bleeding after modeling.

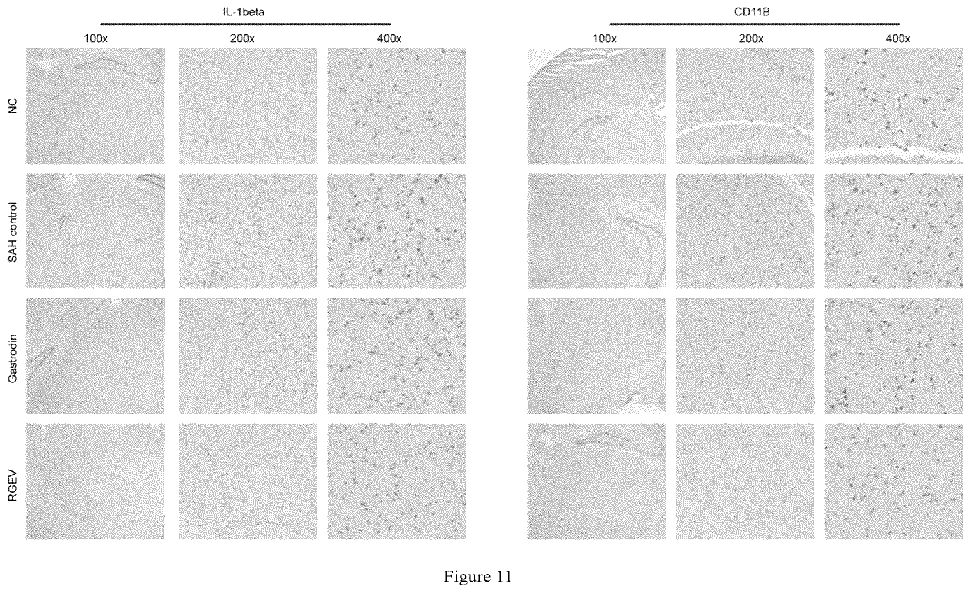

Embodiment 5: Effects of RGEV on IL-1beta expression in brain tissue of SAH model rats and changes in macrophages (using CD11B for marking). (1) Experimental subjects: brain tissue of rats in each group. (2) Experimental grouping: \textcircled{1} NC group; \textcircled{2} SAH surgical model group; \textcircled{3} Gastrodin+SAH surgical model group; \textcircled{4} RGEV+SAH; (3) Experimental steps: 3.1 Tissue sampling; 3.1.1 Use chloral hydrate to anesthetize and kill the rat; 3.1.2 Quickly dissect the rat and take out the brain tissue; use tissue scissors to cut part of the tissue, wash it in PBS and freeze it to −80° C. is reserved for WB use, and part of it is soaked in 10% neutral formalin for fixation. There should be a sufficient amount of fixative, generally more than 10 times the volume of the tissue block; 3.1.3 The fixation time is generally 4-6 hours for small tissues and 24 hours or more for large tissues; 3.1.4 The cutting surface of the material should be smooth and the thickness should be 2-3 mm. If the wax block is too thick, it will dehydrate; 3.1.5 For organizations with special requirements, it is necessary to ensure that the target organization is completely retained when collecting materials. 3.2 Tissue dehydration. 3.2.1 Use an automatic dehydrator to dehydrate the fixed tissue; 3.2.2 The temperature of the automatic dehydrator is set to 60° C.; 3.2.3 Pure water 1 h; 3.2.4 70% ethanol 1 h; 3.2.5 85% ethanol 1 h; 3.2.6 Absolute ethanol 1 h×2 times; 3.2.7 TO 45 min×2 times; 3.2.8 Paraffin 1 h×2 times; 3.2.9 Paraffin 2 h. 3.3 Wax block embedding. The dehydrated tissue needs to be further embedded in wax blocks. The steps are as follows: 3.3.1 The tweezers used must be heated in the embedding machine. Cold tweezers cannot be used, otherwise the tissue will stick to the tweezers; 3.3.2 Quickly take the tissue block with forceps and put it into the paraffin of the embedding frame. Place it with the cut surface facing down in the middle of the bottom of the frame and gently flatten it to ensure that there are no bubbles in the paraffin; 3.3.3 The temperature of the paraffin during embedding should not be too high. The temperature of the embedding machine should be adjusted to about 60° C. Because the temperature will be too high, the tissue will become stiff. After the tissue becomes stiff, curling, shrinkage and deformation will occur. This will It will affect sectioning, staining and result interpretation. After the tissue is taken out of the retort, it must be immersed in the paraffin heated by the embedding machine, so that the temperature of the tissue itself and the embedding paraffin are consistent. If the two temperatures are inconsistent, it may cause the tissue to detach from the surrounding paraffin, thus failing to achieve the goal. to the embedding effect; 3.3.4 When embedding, the side with a flat cut surface and lesions should be placed underneath. If the tissue is uneven, you can use tweezers to gently flatten the tissue; 3.3.5 If the same type of tissue is to be embedded in the same wax block, attention should be paid to laying the tissue flat and keeping the direction consistent to facilitate sectioning. 3.4 Immunohistochemistry: 3.4.1 The steps of slicing, spreading, baking and dewaxing are the same as those of HE stain. Same as above. It should be noted that the glass slides used for spreading should be protected from dewaxing. Because the immunohistochemistry steps are cumbersome, anti-detachment slides are needed to prevent tissue from falling off; 3.4.2 Antigen retrieval: After deparaffinization, place the sections in distilled water for 2 min×2 times, and then place them in PBS for 2 min×3 times. Finally, the slices are immersed in a pressure cooker filled with citrate buffer, that is, the repair solution. After the repair solution in the pressure cooker boils, cover the lid and wait for high-pressure continuous air injection for 3 minutes. Then take the pressure cooker to the ground and wait for it to cool naturally; 3.4.3 Block endogenous peroxidase activity: After the repair solution is naturally cooled, take out the slide rack and put it into PBS for 2 min×3 times. Then put in preheated 3% hydrogen peroxide methanol solution and incubate for 7 minutes; 3.4.4 Blocking: After incubation, take out the slide and put it into PBS solution for 5 min×3 times. Dry the moisture around the tissue on the slide, add an appropriate amount of 10% BSA solution to completely cover the tissue, place it in a 37° C. oven, and seal it for 1 hour; 3.4.5 Incubation primary antibody: After closure, circle the tissue with an IHC pen: take out a slide, dry the liquid around the tissue, and then wipe the liquid on the back of the slide with a paper towel, and then circle the tissue with an IHC pen, taking care not to touch the tissue, the circle drawn is slightly larger than the tissue. Then wash with PBS, 2 min×3 times, absorb the sealing liquid around the tissue as far as possible, prepare the primary antibody, the whole process is carried out on the ice, calculate the amount of antibody and antibody diluent, each specimen is about 50 μL, add the antibody diluent in the EP tube, centrifuge the antibody after removal, then add the antibody with the pipette, and mix it well with the vortex. Add a primary antibody, about 50 μL, do not exceed the circle range when adding, try to add on the tissue so that the entire liquid surface is convex. Negative control was added about 50 ul PBS at the same time, and then put in the refrigerator at 4° C. 12 h; 3.4.6 Incubate the secondary antibody: The next day, take out the slide to observe the presence of the primary antibody, clean the slide once with double-distilled water, and wash the negative control slide and the slide with the antibody dripped separately. Wash with PBS for 2 min Place the slices in a wet box, add secondary antibody dropwise, about 50 μL for each specimen, do not exceed the circled range when adding, and try to make the entire liquid surface added to the tissue appear convex. Then incubate at 37° C. for 20 minutes; 3.4.7 DAB color development: take out the wet box after the secondary antibody incubation is completed, observe the presence of the secondary antibody, wash once with double-distilled water, and wash the negative control slide and the slide with the antibody dripped separately. Then wash with PBS, 2 min×3 times. Dry the water around the washed sliced tissue as much as possible, and add DAB chromogenic solution dropwise, about 50 μL for each specimen. Do not exceed the circled range when adding and try to add it to the tissue. Control the color development time under a light microscope. When brown deposits are found in the tissue, rinse with tap water to terminate the color reaction; 3.4.8 Counterstaining: After terminating dyeing, wash with double-distilled water for 1 min, and then dye with hematoxylin solution. The specific dyeing time depends on the dyeing situation. After the dyeing is completed, rinse with tap water, and then differentiate with 1% hydrochloric acid alcohol for 2 s. Then rinse with tap water for 3 seconds and soak in PBS for 5 minutes to return to blue; 3.4.9 Dehydration: It is the same as the dehydration step in the above-mentioned HE stain and will not be repeated here; 3.4.10 Sealing: After dehydration, place the slices in a fume hood to dry naturally, and seal them with neutral gum; 3.4.11 Photographing and statistics: Observe the tissue morphology and positive expression of the slices under an optical microscope and use Image Pro Plus software to analyze and count the staining results. Result interpretation standard: Immunohistochemistry staining results should consider two aspects: 1. The intensity of positive staining. Positive staining means that the cytoplasm, nucleus or cell membrane shows brownish-yellow sediment signals. The intensity score of positive staining is 0-4 points: no brown-yellow deposit is negative, 0 points; brown-yellow deposit color is weak, 1 point; brown-yellow deposit color is medium, 2 points; brown-yellow deposit color is deep, strong and positive, 3 points. 2. Proportion of positively stained cells. The proportion of positively stained cells is scored as 0-4 points: <5% is 0 points; 5%-25% is 1 point; 26%-50% is 2 points; 51%-75% is 3 points; >75% for 4 points. The product of the intensity of positive staining and the proportion of positively stained cells is called the staining index, with a score of 0-12. The staining index was used for data analysis, and it was considered that 0-3 points were no expression, 4-7 points were low expression, and 8-12 points were high expression. Scoring when staining is uneven: Each score is independent and the results are combined. For example, a section contains 24% of cells with high-intensity staining (1*3=3 points), and the other 74% of cells with weak-intensity staining (3*1=1 point). The final result is 3+1=4, low expression. The experimental results are shown in FIG. 11: the histochemical results of IL-1beta can be drawn. IL-1beta in the modeling group showed a significant increase in expression, and the staining of the brain tissue showed multiple spots and deep staining; The expression of IL-1beta in the gastrodin administration group was down-regulated compared with the model control group. The expression of IL-1beta in RGEV was significantly down-regulated compared with the surgical model and the gastrodin group. However, it was still increased compared with NC, which is also consistent with the general pattern of brain tissue. The results fed back from anatomical structures are the same; CD11B is a widely expressed site of macrophages and is often used to label macrophages. It can be clearly seen from the histochemical results that macrophages are less enriched in normal brain tissue. After SAH, macrophages in the brain tissue were significantly increased and enriched; brain tissue given gastrodin and RGEV in advance can reduce the recruitment and enrichment of CD11B macrophages, and RGEV has the effect of inhibiting macrophage enrichment. Significantly better than gastrodin. The above-mentioned embodiments are preferred embodiments of the present invention, but the embodiments of the present invention are not limited by the above-mentioned embodiments, and any other changes, modifications, and substitutions may be made without departing from the spirit and principles of the present invention, combination, simplification, All should be equivalent substitutions, and all are included in the protection scope of the present invention.

What is claimed is:

1. Application of Gastrodia-derived nanocellular extracellular vesicles in the preparation of drugs for the prevention and/or treatment of subarachnoid hemorrhage.

2. The application of claim 1, wherein: the diameter of the Gastrodia derived nanocellular vesicles is 50-250 nm.

3. The application of claim 1, wherein: the drug comprises a therapeutic effective amount of Gastrodia-derived nanocellular vesicles.

4. Application of Gastrodia-derived nanocellular vesicles in the preparation of products that promote microglial transformation to the M2 phenotype and/or inhibit the transformation of microglia to the M1 phenotype.

5. Application of Gastrodia-derived nanocellular extracellular vesicles in the preparation of products that inhibit subarachnoid hemorrhage-induced microglial activation.

6. The application of claim 4, wherein the product is a drug.

7. The application of claim 1, wherein the Gastrodia-derived nanocellular vesicles are isolated from Gastrodia.

8. The application of claim 5, wherein the product is a drug.
